SEQUENCE LISTING (4 sequences)

SEQ ID NO: 1 - protein, 440 aa, artificial sequence
  Artificial protein fusing the extracellular domain of the protein HveM of the mouse and the crystallisable fragment of the human immunoglobulin G1
MEPLPGWGSAPWSQAPTDNTFRLVPCVFLLNLLQRISAQPSCRQEEFLVGDECCPMCNPGYHVKQVCSEHTGTVCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSDPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 581 aa, artificial sequence
  Artificial protein fusing the extracellular domain (domains V-C-C) of the protein HveC of the pig and the crystallisable fragment of the human immunoglobulin G1
MARMGLAGAAGRWWGLALGLTAFFLPGAHTQVVQVNDSMYGFIGTDVVLHCSFANPLPGVKITQVTWQKATNGSKQNVAIYNPAMGVSVLAPYRERVEFLRPSFTDGTIRLSRLELEDEGVYICEFATFPAGNRESQLNLTVMAKPTNWIEGTQAVLRAKKGKDDKVLVATCTSANGKPPSVVSWETHLKGEAEYQEIRNPNGTVTVISRYRLVPSREDHRQSLACIVNYHMDRFRESLTLNVQYEPEVTIEGFDGNWYLQRMDVKLTCKADANPPATEYHWTTLNGSLPKGVEAQNRTLFFRGPINYSMAGTYICEATNPIGTRSGQVEVNITEFPYTPSPPEHADPEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 376 aa, artificial sequence
  Artificial protein fusing the V domain of the protein HveC of the pig and the crystallisable fragment of the porcine immunoglobulin G1
MARMGLAGAAGRWWGLALGLTAFFLPGAHTQVVQVNDSMYGFIGTDVVLHCSFANPLPGVKITQVTWQKATNGSKQNVAIYNPAMGVSVLAPYRERVEFLRPSFTDGTIRLSRLELEDEGVYICEFATFPAGNRESQLNLTVMGSVGIHQPQTCPICPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTLTCLVIGFYPPDIHVEWKSNGQPEPENTYRTTPPQQDVDGTFFLYSKLAVDKARWDHGDKFECAVMHEALHNHYTQKSISKTQGK

SEQ ID NO: 4 - protein, 578 aa, artificial sequence
  Artificial protein fusing the extracellular domain (domains V-C-C) of the protein HveC of the pig and the crystallisable fragment of the porcine immunoglobulin G1
MARMGLAGAAGRWWGLALGLTAFFLPGAHTQVVQVNDSMYGFIGTDVVLHCSFANPLPGVKITQVTWQKATNGSKQNVAIYNPAMGVSVLAPYRERVEFLRPSFTDGTIRLSRLELEDEGVYICEFATFPAGNRESQLNLTVMAKPTNWIEGTQAVLRAKKGKDDKVLVATCTSANGKPPSVVSWETHLKGEAEYQEIRNPNGTVTVISRYRLVPSREDHRQSLACIVNYHMDRFRESLTLNVQYEPEVTIEGFDGNWYLQRMDVKLTCKADANPPATEYHWTTLNGSLPKGVEAQNRTLFFRGPINYSMAGTYICEATNPIGTRSGQVEVNITEFPYTPSPPEHGSVGIHQPQTCPICPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTLTCLVIGFYPPDIHVEWKSNGQPEPENTYRTTPPQQDVDGTFFLYSKLAVDKARWDHGDKFECAVMHEALHNHYTQKSISKTQGK

(19) United States
(12) Patent Application Publication
(10) Pub. No.: US 2006/0272040 A1
(43) Pub. Date: Nov. 30, 2006

(54) Method for producing a mammal provided with resistance to an alpha-herpes virus mediated infection and mamal obtained by implementing said method and said mamal's progeny

(76) Inventors: Etsuro Ono, Sapporo (JP); Toshimitsu Uede, Sapporo (JP)

Correspondence Address: BAKER & DANIELS LLP;111 E. WAYNE STREET, SUITE 800, FORT WAYNE, IN, 46802, US

(21) Appl. No.: 10/530,539

(22) PCT Filed: Oct. 14, 2003
(86) PCT No.: PCT/FR03/03024

§ 371 Date: Sep. 19, 2005

(30) Foreign Application Priority Data
Oct. 15, 2002 (FR) 0212775

(51) Int. Cl. A01K 67/027; C12N 15/86

(52) U.S. Cl. 800/15; 435/456

(57) ABSTRACT

The invention concerns a method for producing a mammal belonging to a non-human species made resistant by germinal transgenesis to an alpha-herpes virus mediated infection, for which the HveC or nectin-1 polypeptide constitutes a functional receptor. The method consists in introducing by insertion or homologous recombination into the genome of the cells constituting the mammal's germinal line, a transgene enabling expression of a chimeric protein consisting of an extracellular domain of nectin-1 or HveC or one of its parts and an immunoglobulin crystallzable fragment, in a suitable expression system.

Different viruses of the herpes type which are distinguished by their genome and their biological characteristics are known.

A subfamily of these viruses corresponds to the alphaherpesvirus, examples of which include the human herpes simplex virus type 1 and type 2 (HSV-1 and HSV-2), the Aujeszky's disease virus or Pseudorabies virus (PRV) and bovine herpes virus type 1 (BHV-1).

All these viruses have the characteristic of being neurotropes and having a very short replication cycle and a broad host spectrum.

Infection by these viruses causes lesions of the epidermis, situated as a rule in the mucous membranes, followed by spreading of the virus to the nervous system which can involve acute inflammations and latent infections.

Examples of the alphaherpesviruses involving the greatest damage are the PRV virus which is a pathogenic agent of major economic importance in pig production due to the direct cost of the pathologies caused and thus to the means for combating it employed.

This virus is widely present in the majority of regions of high pig production (Europe, North America and Asia).

There are currently several vaccines against the PRV virus which represent a major market worldwide.

Such a vaccination is not however without disadvantages considering in particular the cost involved in the need to vaccinate a large proportion of animals in a herd and practical problems linked with this which make this operation particularly awkward, all the more so because in general it is necessary to carry out several injections.

The cost and constraints of this operation can limit its use and thus even also its effectiveness.

Strategies for eradicating the virus are also used with variable but always weak results.

The BHV-1 virus responsible for infectious bovine rhinotracheitis again causes major problems in farming.

This virus is in fact highly contagious both in new-born calves and in older animals; it can cause inflammation of the nasal cavity and the larynx, ocular lesions and respiratory problems, but can also occur in the brain causing encephalitis or even take on genital forms.

These different problems which are frequently fatal in the new-born calf mean major losses for farmers; moreover, in dairy cattle a dramatic fall in milk production can be observed.

There are also several vaccines against the BHV-1 virus, but vaccination has the same disadvantages as vaccination against the PRV virus in pig farming.

Furthermore, vaccines against the BHV-1 virus injected by intra-muscular administration have been considered responsible for abortions in pregnant cows.

Eradication of the virus has also been proposed in particular in some European countries, but it has proved particularly difficult due to the latent character of the virus which can remain inactive within the organism for a long time before actually manifesting itself, particularly as a result of stress.

Consequently, the conception of a process allowing lines of transgenic pigs to be produced constitutively resistant to the PRV virus or even lines of transgenic cows constitutively resistant to the BHV-1 virus would be of considerable economic interest.

The object of the invention is to propose such a process.

This has been possible due to previous research carried out on a mouse model which, just like the pig or the cow, is sensitive to certain alphaherpesviruses and in particular to the HSV1 and PRV viruses.

It is known that the alphaherpesviruses are linked to the cells first of all thanks to the interaction of a viral glycoprotein gC, entering the constitution of the virion and heparan sulphate membrane present on the surface of the cells, whilst subsequent fusion between the virion envelope and the cell membrane then produces other glycoproteins (gB, gD, gH and gL).

Numerous works have focussed on the study of potential receptors of the alphaherpesviruses present on the surface of the cells of host mammals having a fixing ability of the viral particle and possibly being able to thus neutralise its infectious power.

Four receptor proteins of the alphaherpesviruses have been identified to date, namely:
HVEM or HveA which is an entry mediator for the HSV1 and HSV2 viruses but not the PRV virus (Montgomery, R. I., Warner, M. S., Lum B. J., and Spear, P. G. (1996). Herpes simplex virus-1 entry into cells mediated by a novel member of the TNF/NGF receptor family. Cell 87, 427-436).
three members of the immunoglobulin superfamily (HveB or nectin-2; HveC or nectin-1 and HveD)

(Cocchi, F., Menotti, L., Mirandola, P., Lopez, M., and Campadelli-Fiume, G. (1998). The ectodomain of a novel member of the immunoglobulin subfamily related to the poliovirus receptor has the attribute of a bona fide receptor for herpes simplex virus types 1 and 2 in human cells. J. Virol. 72, 9992-10002;

Geraghty, R. J., Krummenacher, C., Eisenberg, R. J., Cohen G. H., and Spear, P. G. (1998) Entry of alphaherpesviruses mediated by poliovirus receptor related protein 1 and poliovirus receptor. Science 280, 1618-1620;

Shukla, D., Liu, J., Blaiklock, P., Shworak, N. W., Bai, X., Esko, J. D., Cohen, G. H., Eisenberg, R. J., Rosenberg, R. D., and Spear, P. G. (1999). A novel role for 3-O-sulfate heparan sulfate in herpes simplex virus entry. Cell 99, 13-22;

Warner, M. S., Martinez, W., Geraghty, R. J., Montgomery, R. I., Witbeck, J. C., Xu, R., Eisenberg, R. J., Cohen, G. H., and Spear, P. G. (1998). A cell surface protein with herpesvirus entry activity (HveB) confers susceptibility to infection by herpes simplex virus type 2, mutants of herpes simplex virus type 1 and pseudorabies virus. Virology 246, 179-189.

According to the publications Spear, P. G. (1993). Entry of alpha-herpesviruses into cells. Semin. Virol. 4, 167-180; Campadelli-Fiume, G., Arsenakis, M., Farabegoli, F., and Roizman, B. (1988). Entry of herpes simplex virus 1 in BJ cells that constitutively express viral glycoprotein D is by endocytosis and results in degradation of the virus. J. Virol. 62, 159-167, it has been suggested that in addition to the initial link with the heparan sulphate, it is the interaction of the glycoprotein gD of the alphaherpesvirus with a receptor present on the surface of the cell which allows entry of the virus in infectious form and that in certain types of cells, the HSV-1, PRV and BHV-1 viruses can use a common receptor of the glycoprotein gD to enter the cell.

It has therefore been proved that the protein HVEM can allow entry of the HSV1 and HSV2 viruses in the non-permissive cells, but not that of the PRV virus.

On the other hand, it has been demonstrated that the protein HveC, and particularly that of the pig, behaves like a functional receptor not only of the HSV. 1 virus but also of the animal alphaherpesviruses PRV and BHV-1 (Milne, R. S. B., Connolly, S. A., Krummenacher, C., Eisenberg, R. J., and Cohen, G. H. (2001). Porcine HveC, a member of the highly conserved HveC/nectin 1 family, is a functional alphaherpesvirus receptor. Virology 281, 315-328; Cocchi, F., Menotti, L., Mirandola, P., Lopez, M., and Campadelli-Fiume, G. (1998). The ectodomain of a novel member of the immunoglobulin subfamily related to the poliovirus receptor has the attribute of a bona fide receptor for herpes simplex virus types 1 and 2 in human cells. J. Virol. 72, 9992-10002; Geraghty, R. J., Krummenacher, C., Eisenberg, R. J., Cohen G. H., and Spear, P. G. (1998) Entry of alphaherpesviruses mediated by poliovirus receptor related protein 1 and poliovirus receptor. Science 280, 1618-1620).

The abilities to bind to the viral glycoprotein gD are more particularly attributed to the V domain for HveC and to the first two cystein-rich domains (“CRD”) for HVEM (Structure-Based Analysis of the Herpes Simplex Virus Glycoprotein D Binding Site Present on Herpesvirus Entry Mediator HevA (HVEM). Connolly S A, Landsburg D J, Carfi A, Wiley D C, Eisenberg R J, Cohen G H; J virol 2002, Nov. 1; 76 (21): 10894-10904).

However, it has been shown in the publication by Martinez W M, Spear P G, Amino acid substitutions in the V domain of nectin-1 (HveC) that impair entry activity for herpes simplex virus types 1 and 2 but not for Pseudorabies virus or bovine herpesvirus 1, J Virol. 2002 July; 76 (14): 7255-62, that the abilities to bind with the viral glycoprotein gD of the protein HveC could be significantly changed without eliminating the abilities of this protein to trigger fusion of the cellular and viral membranes and thus allow entry of the PRV and BHV-1 viruses in the cell in an infectious form.

It has also been shown in the publication by Campadelli-Fiume G, Cocchi F, Menotti L, Lopez M. the novel receptors that mediate the entry of herpes simplex viruses and animal alphaherpesviruses into cells Rev Med Virol. 2000 September-October; 10 (5): 305-19, that the protein HveC expressed by the mouse can play a mediator role in the entry of the alphaherpesviruses PRV, BHV-1 and HSV independently of a detectable interaction with the glycoprotein gD.

It has moreover been globally concluded in the study by Geraghty R J, Fridberg A, Krummenacher C, Cohen G H, Eisenberg R J, Spear P G, use of chimeric nectin-1 (HveC)-related receptors to demonstrate that ability to bind alphaherpesvirus gD is not necessarily sufficient for viral entry, Virology. 2001 Jul. 5; 285 (2): 366-75, that the interaction of the protein HveC with the glycoprotein gD was not sufficient to allow entry of the virus in the cell and that the characteristics of the protein HveC responsible for the mediator properties for entry of the HSV, PVR and BHV1 viruses in the target cells could not be reduced to its abilities to bind with the glycoprotein gD.

It should furthermore be noted that the protein HveC has a remarkably well conserved polypeptide sequence between the mammal species; by way of example, 97% of the amino acids are common to the protein HveC expressed by the pig and the protein HveC expressed in cows, which implies a strong identity of structure and function in these two species.

According to the publication by INOBE MANABU et al “Functional analysis of HVEM, a member of TNFR family, by using a transgenic mice expressing soluble form of HVEM” (Biosciences information Service, Philadelphia, Pa.-US-2001.03.07), lines of genetically modified mice have been created for experimental purposes by introducing in the genome of these mice a coding transgene for a chimeric protein composed of the extracellular domain of the protein HVEM and the crystallisable portion Fc of the immunoglobulin IgG, in order to study the implication of the protein HVEM in the regulation of the immune system.

It has thus been confirmed that the expression of a soluble form of HveM, a member of the family of the receptor of TNFalpha, produces an immunosuppressor effect.

From this previous knowledge, according to the invention a process for producing a mammal belonging to a non-human species rendered resistant by germinal transgenesis to infection by an alphaherpesvirus for which the polypeptide HveC or nectin 1 constitutes a functional receptor, is proposed, characterised in that a transgene allowing the expression of a chimeric protein composed on the one hand of the extracellular domain of nectin-1 or HveC or one of its parts and on the other the crystallisable fragment of an immunoglobulin, in particular a gamma type immunoglobulin, is introduced by insertion or homologous recombination in the genome of the cells comprising the germinal line of the mammal or one of these ancestors, in an appropriate expression system.

The idea on which the invention is based therefore consisted of using in part the mediator abilities of the protein HveC in relation to the entry of the targeted virus, but in a harmless way for the cell (by isolating its cellular domain or one of its parts) so as to ultimately inhibit the entry of this virus into the cell and favour its elimination, by a process which is still to be determined.

The action mechanism of the chimeric protein expressed could in particular comprise, thanks to the fixing ability of this both for the viral particle and the cellular receptor Fc, an increase in the phagocytosis and destruction abilities of the virions by the macrophages and dendritic cells and an activation of the NK cytotoxic lymphocytes.

It could also comprise the formation of membrane receptors of the alphaherpesviruses in the form of functionally modified multimers by incorporating one or several units of the chimeric protein, allowing the viral particles to be fixed to the surface of the cell but not allowing them to enter the cytoplasm in an infectious form.

According to the invention, the protein HveC or nectin-1 and/or the immunoglobulin belong preferably to the homologous species.

Also only part of the nectin-1 or HveC could be used, or even, if necessary, the mutated forms of these parts, selected for their ability to bind with the targeted virus.

The first stage of the process according to the invention therefore corresponds to the preparation of the transgene which can be carried out using methods well-known by people skilled in the art and abundantly described in the literature consisting of cloning:

On the one hand, either complementary DNA of the RNA transcribed for the cellular receptor gene from an RNA preparation extracted from a tissue sample taken on a mammal, or the chromosome region (all exons and introns) comprising the gene of this receptor from a genome DNA preparation also extracted from a tissue sample taken from a mammal, or a chimeric construction constituted for part of the cDNA and for the remaining part of the polypeptide chain of the corresponding genome fragment (“mini-gene”).

In all cases, only the part corresponding to the extracellular domain of this receptor will be used, or in another version of the process, a sub-part of this domain or even, where necessary, a polypeptide sequence essentially derived from this extracellular domain.

On the other hand, either the complementary cDNA of the RNA transcribed for one of the heavy chain genes for the class and sub-class of immunoglobulin selected (for example G1) from an RNA preparation extracted from a tissue sample taken from a mammal, or the chromosome region (all exons and introns) comprising this heavy chain gene from a genome DNA preparation also extracted from a tissue sample taken from a mammal, or a chimeric construction composed for part of the cDNA and for the remaining part of the polypeptide chain of the corresponding genome fragment (“mini-gene”). The construction undertaken will advantageously retain the coding regions for the Hinge, CH₂ and CH₃ domains only of the heavy immunoglobulin chain selected (crystallisable fraction).

An example of such a construction for the human immunoglobulin G1 is described in the publication CTLA-4 Is a Second Receptor for the B Cell Activation Antigen B7. By Peter S. Linsley, William Brady, Mark Urnes, Laura S. Grosmaire, Nitin K. Damle, and Jeffrey A. Ledbetter; J. Exp. Med. © The Rockefeller University Press; volume 174, September 1991, 561-569.

The cloning operations will be carried out from existing previous knowledge relating to the genes used, that is their sequence, their chromosome localisation, if possible in the homologous species, but being based on the known sequences for this gene in other mammals.

These operations could be carried out by a polymerisation chain reaction (PCR) preceded by a reverse transcription stage for the complement DNA or by detection within banks of genome DNA of the targeted species of the clones likely to be hybridised with a specific probe or to produce a specific PCR amplicon of the gene researched.

This construction will be carried out so as to join the coding sequences for the extracellular domain of nectin-1 or HveC or one of its parts at 5′ of the coding sequences for the crystallisable fragment of the heavy immunoglobulin chain (terminated by a stop codon) whilst complying with the original reading framework of the two genes, and possibly the nature and effectiveness of the intron-extron junctions if they have been included, so as to ultimately ensure the expression of a chimeric protein constituted for its terminal amino part of the polypeptide corresponding to the extracellular domain of the HveC cellular receptor or one of its sub-parts and for its terminal carboxy part of the Hinge, CH2 and CH3 domains of the heavy immunoglobulin chain.

This construction will be undertaken in an expression vector allowing a strong expression of the chimeric protein in one or several biological compartments of the host where it will allow protection of the cells so as to render the host globally resistant to the initial infection or to its development. The process will consist in particular of using active expression systems either constitutively in all the cells or more specifically in the target tissues of the viral infection such as the tissues of the central nervous system or the epithelial tissues (in particular those of the respiratory system).

The expression vector could comprise a promoter region, a termination signal, stimulator elements of the transcription, isolator sequences of the chromatin context, other transcription units and all elements likely to ensure the desired expression.

The process will consist advantageously of using expression systems constituted from cloned regulator sequences in the homologous species, or for application to animals for production, in other animals normally reared for human consumption.

The second stage of the process according to the invention consists of introducing the transgene thus obtained in the genome of the cells comprising the germinal line of the targeted host mammal by insertion or homologous recombination, again by a method well-known to persons skilled in the art such that this transgene is integrated in the genetic inheritance of this mammal.

Pronuclear micro injection of the DNA segment encoding the transgene or nuclear transfer of cells transformed in culture by the transgene in particular can be used.

The invention also relates to a mammal belonging to a non-human species rendered resistant by germinal transgenesis to an infection by an alphaherpesvirus for which the polypeptide HveC or nectin-1 constitutes a functional receptor by the effect of the expression of a chimeric protein composed on the one hand of the extracellular domain of the nectin-1 or HveC or one of its parts preferably of the homologous species, and on the other of the crystallisable fragment of an immunoglobulin, particularly of a gamma type immunoglobulin preferably of the homologous species.

The invention also relates to the progeny of such a mammal, having inherited by descent the transgene inserted in the genome of the germinal line of one of its parents.

According to the invention, the alphaherpesvirus can advantageously be the PRV virus and the mammal belong to the porcine species.

According to a variation of the invention, the alphaherpesvirus can also be the BHV-1 virus and the mammal belong to the bovine species.

It is essential according to the invention that the transgenesis operation is a germinal transgenesis such that the progeny of the mammal are also likely to express the chimeric protein.

The invention also relates to a genetic material such as the semen or ooecytes or embryos essentially derived from transgenic mammals of the above-mentioned type.

Several examples of sequences of chimeric proteins according to the invention are attached.

These are sequences of amino acids which comprise the signal peptide at the terminal amino end which will be processed during maturation.

A chimeric protein according to this model is also described in the document JP-2001-328430 within the framework of another application.

The feasibility of the process according to the invention has been confirmed by

1) experimental results obtained within the framework of the in vivo analysis of the resistance to the human herpes simplex virus type 1 (HSV-1) of transgenic mice expressing a chimeric protein Hvem—Ig.

According to these tests, a coding transgene for a chimeric protein composed of the extracellular domain of the murine receptor HVEM of this virus and the crystallisable portion Fc of the human immunoglobulin IgG-1 was introduced by germinal transgenesis into the DNA of these mice.

The extracellular murine HveM domain was cloned by RT PCR from a preparation of RNA extracted from female rat cells stimulated by concavaline A, obtained on stock mice BALB/c.

The primers used for the RT PCR reaction were 5′-TAACTCGAGCTCTTGGCCTGAAGTTTC-3′ and 5′-TTAAGGATCCGAGGAGCAGGTGGTGTCT-3′.

The cDNA was inserted in the Xhol and BamHl restriction sites of a plasmid having the sequence of the crystallisable fragment of the human immunoglobulin G1 (as described in the publication by Nakagawa I., Murakami, M., Ijima, K., Chikuma, S., Saito, I., Kanegae, Y., Ishikura, H., Yoshiki, T., Okamoto, H., Kitabatake, A., and Uede, T. (1998). Persistent and secondary adenovirus-mediated hepatic gene expression using adenovirus vector containing CTLA41gG. Human Gene Therapy 9, 1739-1745).

The XhoI/XbaI fragment containing the DNA encoding the chimeric protein HveMIg was isolated from this construction and inserted in turn, after blunt-ending, in the SwaI restriction site of the cosmid vector pAxCAwt (commercially distributed by the Company TAKARA, Kyoto, Japan) under the control of the CAG promoter (β actin promoter) known to allow a high constitutive expression in any type of cell (Niwa, H., Yamamura, K., and Miyazaki, J. (1991). Efficient selection for high-expression transfectants with a novel eukaryotic vector. Gene 108, 193-200).

Three lines A, B, C of transgenic mice each from an independent founder expressing this chimeric protein HVEM Ig, were then created by microinjection of the Pmel/Sall fragment of this vector containing the stimulation factor of the viral gene CMV IE transcription, the promoter of the Beta actin gene of the chicken, the sequence of the protein HVEMIg and the polyadenylation 3′ signal of the Beta globin locus of the rabbit, in the fertilised embryo pronuclei of mice (genotype F1 C57 BL/6×SJL).

The presence of the protein HVEMIg was detected as a specific band revealed by an anti-HVEMIg antibody (produced by hyperimmunisation on the rabbit) by immunoelectrophoresis in the serum of the three lines of transgenic mice, with a lower average content for the mice of line B.

The construction carried out is shown schematically in FIG. 1.

The concentration of chimeric protein HVEMIg in the serum of the mice of the three transgenic lines A B C is shown in Tables 1, 2, 3 and 4 attached.

The transgenic mice of three lines developed normally and no differences were noted between the weights of these mice and those of their non-transgenic homologs from the same litter.

To determine whether the transgenic mice expressing the chimeric protein HVEMIg were effectively protected against the HSV-1 virus, transgenic mice and non-transgenic control mice from the same litter were inoculated intravenously and a dose of 10⁹ cfu corresponding to ten times the lethal dose (10 LD 50) of virus.

The LD 50 was determined initially in the less sensitive of the two lines of mice used to produce the hybrid transgenic animals.

According to FIGS. 2, 3 and 4, the transgenic T_{g} mice respectively of lines A B and C and the non-transgenic T_{g} mice still alive up to 14 days after infection were counted.

It could also be noted that all the lines of transgenic mice were resistant to the HSV-1 virus.

More specifically, all the transgenic mice of lines A and C survived inoculation with the virus and remained in good health for several months after the trial (Tables 1 and 4).

Only one transgenic mouse of line B died after inoculation with the virus whilst the other six survived (Table 3), but line B is the one for which the serum rates measured for HVEMIg were the lowest.

On the other hand, six of the seven non-transgenic mice from the same litters as the transgenic mice of line A, 13 of the 14 non-transgenic mice from the same litters as the transgenic mice of line B and six of the seven non-transgenic mice from the same litters as the transgenic mice of line C developed symptoms such as paralysis or died in the 14 days following inoculation with the HSV-1 virus.

The expression of the LAT of the HSV-1 virus in the trigeminal ganglia of the surviving mice after inoculation by the method described in the publications was investigated.
Spivak, J. G., and Fraser, N. W. (1987). Detection of herpes virus type 1 transcripts during latent infections in mice. J. Virol., 61, 3841-3847.
Stevens, J. G., and Cook, M. L. (1971). Latent herpes simplex virus in spinal ganglia of mice. Science 173, 843-845.

The RT-PCR method was thus used to detect the LAT expression.

This was observed in the non-transgenic mice which developed symptoms and in a single transgenic mouse of line B which did not present symptoms, but was not on the other hand observed in any of the other transgenic mice tested nor in the surviving non-transgenic mice which did not develop symptoms.

Within the framework of this study, a control trial was also carried out in order to determine whether the transgenic mice expressing the chimeric protein HVEMIg were protected against the PRV virus when the protein HVEM is not a functional receptor for the PRV virus.

Transgenic mice of line A and non-transgenic mice from the same litter were for that purpose inoculated intravenously with a dose corresponding to 10 times the lethal dose (10 LD 50) of PRV virus (Table 2 and FIG. 5).

It could also be noted that with the exception of a transgenic mouse that survived for 10 days, all the mice died in the five days following inoculation with the PRV virus.

Subsequently, the transgenic mice expressing the chimeric protein HVEMIg did not prove to be protected against the PRV virus.

Within the framework of this study, it was also sought to determine whether the resistance of the transgenic mice to inoculation with the HSV-1 virus which could be noted in vivo accompanied in parallel a resistance of the cells of these mice once isolated.

Cultures of embryonic fibroblasts of transgenic mice or non-transgenic mice were therefore inoculated with the HSV-1 virus.

The lysis ranges caused by the virus in the cellular culture 5 days after inoculation were then counted and it was noted that the number of these ranges was clearly higher in the case of the fibroblasts of non-transgenic mice than in the case of fibroblasts of transgenic mice (on average 22 plaques per culture disk for the non-transgenic mice and 1 plaque per culture disk for the transgenic mice).

A similar test for the PRV virus was carried out in parallel by inoculating cultures of embryonic fibroblasts from transgenic mice or non-transgenic mice with the PRV virus without noting any significant differences between the number of lysis ranges observed in the transgenic mice and in the non-transgenic mice.

These results are likely to prove that the chimeric protein HVEMIg expressed by the fibroblasts of the transgenic mice is involved in the inhibition of the adsorption of the HSV-1 virus by the embryonic fibroblasts.

In a complementary test, the results of which are shown in Table 5, it was investigated whether the chimeric protein HVEMIg present in the serum of transgenic mice could inhibit the infection of cellular cultures by the HSV-1 or PRV viruses.

Serum was therefore collected from transgenic mice of line C and the HSV-1 virus or PRV virus incubated with an inoculate of this serum prior to bringing it into contact with the Vero cell cultures.

It could thus be established that the serum of transgenic mice of line C can protect Vero cells from contamination by the HSV-1 virus but not from contamination by the PRV virus.

A control test carried out with serum from non-transgenic mice was in contrast not able to note any antiviral activity.

It was moreover noted that the serum from transgenic mice of line C no longer presents any antiviral activity after it was brought into contact with a polyclonal anti-HVEMIg serum produced by hyperimmunisation on the rabbit for 30 minutes at ambient temperature.

This last result confirms that the seroneutralising abilities of the serum of the transgenic mice can be attributed to the expression of the chimeric protein.

All of these results demonstrate that the antiviral activity noted comes from the chimeric protein HVEMIg present in the serum of the transgenic mice, but are also without doubt associated with the expression of this chimeric protein on the surface of the cells of the host just as the embryonic fibroblasts evaluated in these tests.

These results confirm the antiviral effectiveness of the in-vivo expression of a modified form of a membrane receptor of the alphaherpesviruses, despite the immunosuppressive properties described in the case of the protein HveM.

2) experimental results obtained within the framework of the in vivo analysis of the resistance to the PRV virus of transgenic mice expressing a chimeric protein Hvec-Ig.

According to these tests, a coding transgene for a chimeric protein composed of the extracellular domain of the porcine receptor HveC of the PRV virus and the crystallisable portion Fc of the human immunoglobulin IgG-1 was introduced by germinal transgenesis into the DNA of these mice.

The porcine HveC extracellular domain was cloned by RT PCR from an RNA preparation extracted from pig cells.

The primers used for the RT PCR reaction were 5′-TAACTCGAGCTCTTGGCCTGAAGTTTC-3′ and 5′-TTAAGGATCCGAGGAGCAGGTGGTGTCT-3′ as described and following the conditions proposed in the publication (Milne, R. S. B., Connolly, S. A., Krummenacher, C., Eisenberg, R. J., and Cohen, G. H. (2001). Porcine HveC, a member of the highly conserved HveC/nectin 1 family, is a functional alphaherpesvirus receptor. Virology 281, 315-32).

The cDNA was inserted into the Xhol and BamHl restriction sites of a plasmid having the sequence of the crystallisable fragment of the human immunoglobulin G1 (as described in the publication by Nakagawa I., Murakami, M., Ijima, K., Chikuma, S., Saito, I., Kanegae, Y., Ishikura, H., Yoshiki, T., Okamoto, H., Kitabatake, A., and Uede, T. (1998). Persistent and secondary adenovirus-mediated hepatic gene expression using adenovirus vector containing CTLA41gG. Human Gene Therapy 9, 1739-1745). The XbaI/XbaI fragment of this plasmid containing the DNA encoding the HveC-Ig fusion was isolated, blunt-ended and bound to the SalI adaptors, in order to insert it in turn, after digestion by Xho1 and Sal1, in the XhoI restriction site of the vector pCXN2 under the control of the CAG promoter (β actin promoter) known to allow a high constitutive expression in any type of cell (Niwa, H., Yamamura, K., and Miyazaki, J. (1991). Efficient selection for high-expression transfectants with a novel eukaryotic vector. Gene 108, 193-200). The recombinant plasmid thus obtained was designated Pcxn2/pHvecIg.

Six lines #6, #22, #32, #33, #37 and #45 of transgenic mice each from an independent founder expressing this chimeric protein HveC-Ig were created by microinjection of the SalI/SalI fragment of this plasmid containing the stimulation factor of the transcription of the viral gene CMV IE, the promoter of the Beta actin gene of the chicken, the sequence of the protein HVEMIg and the polyadenylation 3′ signal of the Beta globin locus of the rabbit, in fertilised embryo pronuclei of mice (strain C57/BL6).

The construction carried out is shown schematically in FIG. 6.

The transgenic mice of the six lines developed normally and no differences were noted between the weights of these transgenic mice and the weight of the non-transgenic mice from the same litter.

Viral Trials by Intra-Peritoneal Injections

To determine whether the transgenic mice expressing the chimeric protein HveC-Ig were effectively protected against the PRV virus, a dose of 500 p.f.u. corresponding to twenty times the lethal dose (20 LD 50) of virus was inoculated in a first trial series intraperitoneally in transgenic mice and non-transgenic control mice from the same litter.

According to Table 6, the transgenic T_{g} mice respectively from lines #6, #22, #32, #33, #37 and #45 and the non-transgenic T_{g} mice still alive up to 14 days after infection were counted.

It could thus be noted that the 6 lines of transgenic mice were resistant to the PRV virus in this renowned strict trial, with 100% survival of the transgenic animals with the exception of a single animal for line #33.

On the other hand, only around 9% of the non-transgenic mice survived, with a variation according to the trials conducted for each line.

These trials were confirmed for lines #22 and #32 in an independent laboratory using a different strain of the PRV virus.

Viral Trials by Intranasal Infection

In a second series of trials, transgenic and non-transgenic control mice from the same litter were inoculated intranasally with a dose of 250 p.f.u. corresponding to ten times the lethal dose (20 LD 50) of virus.

According to Table 7, the transgenic T_{g} mice respectively from lines #6, #22, #32, #33 and #37 and the non-transgenic non T_{g} mice still alive up to 14 days after infection were counted.

It could thus be noted that the 5 lines of transgenic mice were relatively resistant to the PRV virus in this renowned strict trial, with around 70% survival of the transgenic animals.

On the other hand, only around 10% of the non-transgenic mice survived, with a variation according to the trials conducted for each line.

These results demonstrate an effective protective action of the transgene allowing the expression of the chimeric protein HveC-Ig in the context of a strict trial in the mouse by intranasal administration.

Overall, all these results show the effectiveness in-vivo of the protection conferred to a mammal by the process according to the invention, based on the inhibition of the entry of an alphaherpesvirus in the target cell. This effectiveness is only possible because the process proposed probably allows several levels of action, the mechanisms of which are not all fully explained: in addition to the binding abilities of the viral proteins gD known for the HveM and HveC receptors, the use of the extracellular domain alone allows its receptor abilities to be isolated from the complex biological functions of these trans-membrane proteins in their entry and in particular their aptitude to initiate intracellular signalling cascades.

This allows a priori envisaging of the superexpression of this protein domain without nevertheless amplifying the physiological function and without doubt allows the absence of secondary effects observed for the transgenic animals expressing the chimeric protein.

This probably allows in the context of an infection in vivo:
competition to fix the viral particles infecting chimeric proteins in solution or present on the surface of the cells with the functional membrane receptors of the virus;
neutralisation of the viral particles by opsonisation and increasing of the phagocytosis by the macrophages and dendritic cells;
activation of the cellular immunity by stimulation of the NK lymphocytes.

The effectiveness of the process in the case of experimental infections by intranasal administration together with (for HveMIg) the resistance of embryonic fibroblasts from transgenic animals move towards effective inhibition of the entry of the virus in the sensitive target cells by the membrane fraction of the chimeric protein expressed, beyond the seroneutralising ability of the soluble fraction secreted in the serum.

This inhibition of the entry of the virus in the cell could be the result of modified membrane receptors of the virus according to a dominant method, authorising fixing of the virus to the surface of the target cells but not its entry in the cytoplasm in an infectious form.

The effectiveness of the process on the entry of the virus and the first results relating to the viral latency after infection (in the case of HveM and HSV1) moreover allow the absence of latency in the breeding animals thus protected to be envisaged, which is not necessarily the case for vaccination strategies, and therefore a more effective control of any resurgence of the infection.

3) Experimental results obtained within the framework of the in vitro analysis of the resistance to the BHV-1 and PRV virus of cellular lines transformed by plasmids expressing chimeric proteins constructed from the extracellular domain of the porcine protein HveC and the crystallisable fragment of the human immunoglobulin Ig.

According to this test, lines of Vero cells transformed by plasmids expressing a soluble form of the porcine protein HveC inhibiting entry of the BHV-1 and PRV viruses were prepared and then the resistance of these cellular lines to infection by these viruses was analysed to determine the antiviral properties of the soluble form of the porcine protein HveC in vitro.

To construct a plasmid expressing a soluble form of the porcine protein HveC (PHveCIg) an expression vector PCXN2 was used allowing transcription of a coding messenger RNA for the extracellular domain of the porcine protein HveC together with the crystallisable fragment Fc of the human immunoglobulin IgG1.

The extracellular porcine domain HveC was cloned by RTPCR from an RNA preparation extracted from pig cells.

The primers used for the RT PCR reaction were 5′-TAACTCGAGCTCTTGGCCTGAAGTTTC-3′ and 5′-TTAAGGATCCGAGGAGCAGGTGGTGTCT-3′ as described and according to the conditions proposed in the publication (Milne, R. S. B., Connolly, S. A., Krummenacher, C., Eisenberg, R. J., and Cohen, G. H. (2001). Porcine HveC, a member of the highly conserved HveC/nectin 1 family, is a functional alphaherpesvirus receptor. Virology 281, 315-32).

The cDNA was inserted in the Xhol and BamHl restriction sites of a plasmid having the sequence of the crystallisable fragment of the human immunoglobulin G1 (as described in the publication by Nakagawa I., Murakami, M., Ijima, K., Chikuma, S., Saito, I., Kanegae, Y., Ishikura, H., Yoshiki, T., Okamoto, H., Kitabatake, A., and Uede, T. (1998). Persistent and secondary adenovirus-mediated hepatic gene expression using adenovirus vector containing CTLA41gG. Human Gene Therapy 9, 1739-1745). The XbaI/XbaI fragment of this plasmid containing the DNA encoding the HveC-Ig fusion was isolated, blunt-ended and bound to the SalI adaptors, in order to insert in turn in the XhoI restriction site of the vector pCXN2 under the control of the known CAG promoter (β actin promoter) to allow a high constitutive expression in any type of cell (Niwa, H., Yamamura, K., and Miyazaki, J. (1991). Efficient selection for high-expression transfectants with a novel eukaryotic vector. Gene 108, 193-200). The recombinant plasmid thus obtained was designated Pcxn2/pHvecIg.

Cellular lines transformed in a stable way were prepared by transfection with the plasmid thus obtained.

The cellular lines thus transformed were cultivated under conditions allowing accumulation of the chimeric protein in the medium, that is 24 hours of additional cultures after spreading at subconfluence.

These cultures were then inoculated with the PRV or BHV-1 viruses with a multiplicity of 50 p.f.u. per box of 35 mm diameter with an incubation time of one hour followed by two rinses with DMEM prior to covering with the medium DMEM 0.5% agar.

The lysis ranges caused by the virus in the cellular culture 4 days after inoculation were then counted. It was noted that in the cellular lines expressing the chimeric protein PHveCIg the number of lysis plaques was notably reduced by comparison with the control resistant cellular lines and the Vero lines four days after inoculation.

The results obtained are shown in Table 8.

The cellular lines transformed by the plasmid PCXN2/pHveC Ig are clearly resistant to attack by the PRV and BHV-1 viruses.

4) Experimental results obtained within the framework of the in vitro analysis of the resistance to the BHV-1 and PRV viruses of cellular lines transformed by plasmids expressing chimeric proteins constructed from the extracellular domain of the porcine protein HveC and the crystallisable fragment of the pig immunoglobulin g1.

According to this test, lines of Vero cells transformed by plasmids expressing a soluble form of the porcine protein HveC inhibiting entry of the BHV-1 and PRV viruses were prepared, then the resistance of these cellular lines to infection by these viruses analysed to determine the antiviral properties of the soluble form of the porcine protein HveC in vitro.

To construct a plasmid expressing a soluble form of the porcine protein HveC (PHveCIg) a pCXN2 expression vector was used allowing transcription of a coding messenger RNA for the extracellular domain of the porcine protein HveC and the crystallisable fragment Fc of the porcine immunoglobulin IgG-1.

The porcine HveC extracellular domain was cloned by RT PCR from an RNA preparation extracted from pig cells.

The primers used for the RT PCR reaction were 5′-TAACTCGAGCTCTTGGCCTGAAGTTTC-3′ and 5′-TTAAGGATCCGAGGAGCAGGTGGTGTCT-3′ as described and according to the conditions proposed in the publication (Milne, R. S. B., Connolly, S. A., Krummenacher, C., Eisenberg, R. J., and Cohen, G. H. (2001). Porcine HveC, a member of the highly conserved HveC/nectin 1 family, is a functional alphaherpesvirus receptor. Virology 281, 315-32).

The cDNA thus obtained was linked at 5′ of a coding fragment of cDNA for the crystallisable Fc fragment of the porcine immunoglobulin Ig complying with the original reading framework of the two polypeptides.

The cDNA fragment of porcine immunoglobulin was cloned from an RNA preparation extracted from lymphoid pig tissues from a line of the Large White type (FHO25) by RT PCR using as a trigger TAACTCGAGCTCTTGGCCTGAAGTTTC-3′ and 5′-TTAAGGATCCGAGGAGCAGGTGGTGTCT-3′ according to the conditions proposed in the publication by Simon Musyoka Mwangi, Thomas J. Stabel*, Marcus E. Kehrli Jr, development of a baculovirus expression system for soluble porcine tumor necrosis factor receptor type I and soluble porcine tumor necrosis factor receptor type I-IgG fusion protein, Veterinary Immunology and Immunopathology 86 (2002) 251-254.

The cDNA resulting from the fusion codes for a chimeric protein, the amino acids sequence of which is attached (sequence 4).

The cDNA resulting from the fusion was inserted in the Xhol restriction site of the plasmid pCXN2 under the control of the promoter of the beta actin gene of the chicken associated with the stimulation factor of the transcription of the viral gene CMV IE and the polyadenylation sequence of the beta globin of the rabbit.

The resulting plasmid thus obtained was designated PCXN2/pVCC-pFc.

A restricted version of this plasmid designated PCXN2/pV-pFc was constructed using only the V domain of the protein HveC and the crystallisable fragment Fc of the pig immunoglobulin IgG.

The amino acids sequence of the chimeric protein thus obtained is attached (sequence 3).

Cellular lines transformed in a stable manner were prepared by transfection with the plasmid thus obtained.

The cellular lines thus transformed were cultivated under conditions allowing accumulation of the chimeric protein in the medium, that is 24 hours of additional cultures after spreading at subconfluence.

These cultures were then inoculated with the PRV and BHV-1 viruses with a multiplicity of 50 p.f.u. per box of 35 mm diameter with an incubation time of one hour followed by two rinses with DMEM before covering with the medium DMEM 0.5% agar.

The lysis ranges caused by the virus in the cellular culture three days after inoculation were counted and the findings shown in Table 9.

The cellular lines transformed by the two versions of the transgene expressing a chimeric protein according to the invention are clearly resistant to attack by the BHV-1 and PRV viruses.

TABLE 1
Resistance of transgenic mice of line A
to inoculation with the HSV-1 virus

| | Number of | | | | |
|---|---|---|---|---|---|
| Test | animal | Sex | HVEMIg (ug/ml) | Symptoms | Day of death |
| I | A1 * | M | 14.8 | − | |
| | A2 * | M | 10.0 | − | |
| | A3 * | M | 11.6 | − | |
| | A4 * | F | 7.6 | − | |
| | A5 * | F | 8.3 | − | |
| | A6 * | F | 10.0 | − | |
| | A7 * | F | 24.6 | − | |
| | A8 * | F | 8.5 | − | |
| | A9 * | F | 7.9 | − | |
| | A10 * | F | 7.3 | − | |
| | L1 | M | 1.5 | − | |
| | L2 | M | 1.1 | − | |
| | L3 | M | 1.6 | + | 4 |
| | L4 | M | 0.8 | + | 14 |
| | L5 | M | 1.6 | + | |
| | L6 | F | 0.6 | + | 5 |
| II | A11 * | M | 10.1 | − | |
| | A12 * | M | 15.9 | − | |
| | A13 * | M | 10.5 | − | |
| | A14 * | M | 9.3 | − | |
| | A15 * | F | 7.3 | − | |
| | A16 * | F | 14.0 | − | |
| | A17 * | F | 15.4 | − | |
| | A18 * | F | 14.9 | − | |
| | L7 | M | 0.4 | + | 4 |
| | L8 | M | 0.5 | + | |
| | L9 | F | 0.5 | + | |

TABLE 2
Sensitivity of transgenic mice of line A
to inoculation by the PRV virus

| Number of | | | | Day of |
|---|---|---|---|---|
| animal | Sex | HVEMIg (ug/ml) | Symptoms | death |
| A19 * | M | 41.2 | + | 4 |
| A20 * | F | 19.4 | + | 10 |
| A21 * | F | 29.6 | + | 5 |
| A22 * | F | 24.2 | + | 5 |
| A23 * | F | 20.2 | + | 5 |
| C57BL/6 | M | NT | + | 4 |
| C57BL/6 | M | NT | + | 4 |
| C57BL/6 | M | NT | + | 5 |
| C57BL/6 | M | NT | + | 5 |
| C57BL/6 | M | NT | + | 5 |

TABLE 3
Resistance of transgenic mice of line B
to inoculation by the HSV-1 virus

| | Number of | | HVEMIg | | Day of | |
|---|---|---|---|---|---|---|
| Test | animal | Sex | (ug/ml) | Symptoms | death | RT-PCR |
| I | B1 * | M | 8.5 | − | | − |
| | B2 * | M | 7.8 | − | | − |
| | B3 * | M | 7.7 | − | | − |
| | B4 * | F | 8.1 | + | 11 | |
| | L1 | M | 0.4 | + | 6 | |
| | L2 | M | 0.7 | + | 6 | |
| | L3 | M | 0.5 | + | 5 | |
| | L4 | M | 0.7 | + | 7 | |
| | L5 | F | 0.3 | + | 5 | |
| II | B5 * | M | 5.0 | − | | + |
| | B6 * | M | 6.2 | − | | − |
| | B7 * | F | 4.4 | − | | − |
| | L6 | M | 0.6 | − | | |
| | L7 | M | 0.6 | + | 10 | |
| | L8 | M | 0.5 | + | 7 | |
| | L9 | M | 0.6 | + | | + |
| | L10 | F | 0.4 | + | 7 | |
| | L11 | F | 0.5 | + | 5 | |
| | L12 | F | 0.5 | + | 5 | |
| | L13 | F | 0.5 | + | 5 | |
| | L14 | F | 0.4 | + | 6 | |

TABLE 4
Resistance of transgenic mice of line C
to inoculation by the HSV-1 virus

| | Number of | | HVEMIg | | Day of | |
|---|---|---|---|---|---|---|
| Test | animal | Sex | (ug/ml) | Symptoms | death | RT-PCR |
| | C1 * | M | 20.4 | − | | − |
| | C2 * | M | 14.2 | − | | − |
| | C3 * | F | 19.3 | − | | − |
| | C4 * | F | 18.5 | − | | − |
| | C5 * | F | 24.3 | − | | − |
| | C6 * | F | 21.8 | − | | − |
| | C7 * | F | 24.0 | − | | − |
| | C8 * | F | 20.6 | − | | − |
| | L1 | M | 1.3 | − | | − |
| | L2 | M | 0.9 | + | | + |
| | L3 | M | 1.6 | + | 6 | |
| | L4 | F | 1.3 | + | 5 | |
| | L5 | F | 1.4 | + | 5 | |
| | L6 | F | 1.5 | + | 6 | |
| | L7 | F | 1.6 | + | 5 | |

In Tables 1 to 4, the animals marked with an * are transgenic animals whilst the animals marked with an L are non-transgenic control animals from the same litters.

TABLE 5
Neutralisation of the HSV-1 virus by the chimeric protein
HVEMIg in the serum of transgenic mice of line C.
Inoculation of HSV-1 virus on VERO cells after incubation
with variable concentrations of this serum

Number of lysis ranges observed

| Serum (HVEMIg ug/ml) | HSV-1 | PRV |
|---|---|---|
| (20.4) | 0 | 108.0 ± 8.8 |
| (2.04) | 0 | — |
| (0.20) | 1.7 ± 1.6 | — |
| (0.02) | 34.7 ± 16.2 | — |
| (0.20) + anti HVEMIg | 30.3 ± 6.9 | — |
| Control | 44.0 ± 0 | 107.3 ± 2.9 |

TABLE 6
Trials by intraperitoneal administration (20 LD 50)

| | | Number | | | | |
|---|---|---|---|---|---|---|
| | | of | Number of | | | % |
| | Number | controls | animals | Number of | % | sur- |
| | of | (of the | Tg | controls | survival | vival |
| | animals | same | surviving | surviving | trans- | of |
| | Tg | litters) | at 14 | at 14 | genic | con- |
| Line | tested | tested | days | days | animals | trols |
| PhveCIg#6 | 5 | 10 | 5 | 2 | 100 | 20 |
| PhveCIg#22 | 12 | 12 | 12 | 0 | 100 | 0 |
| PhveCIg#32 | 10 | 7 | 10 | 1 | 100 | 14.2 |
| PhveCIg#33 | 7 | 9 | 6 | 0 | 85.7 | 0 |
| PhveCIg#37 | 10 | 4 | 10 | 1 | 100 | 25 |
| PhveCIg#45 | 3 | 12 | 3 | 1 | 100 | 8.3 |

TABLE 7
Trials by intranasal administration (10 LD 50)

| | | Number | | | | |
|---|---|---|---|---|---|---|
| | | of | Number of | | | % |
| | Number | controls | animals | Number of | % | sur- |
| | of | (of the | Tg | controls | survival | vival |
| | animals | same | surviving | surviving | trans- | of |
| | Tg | litters) | at 14 | at 14 | genic | con- |
| Line | tested | tested | days | days | animals | trols |
| PhveCIg#6 | 8 | 7 | 4 | 0 | 50 | 0 |
| PhveCIg#22 | 23 | 21 | 18 | 2 | 78.3 | 9.5 |
| PhveCIg#32 | 10 | 8 | 6 | 1 | 60 | 12.5 |
| PhveCIg#33 | 17 | 8 | 11 | 1 | 64.7 | 12.5 |
| PhveCIg#37 | 10 | 7 | 9 | 1 | 90 | 14.2 |

TABLE 8
Resistance of transformed cellular lines to the PRV and BHV-1
alphaherpesviruses

| | |
|---|---|
| Cellular | Number of lysis ranges observed |

| line* | PHveCIg | PRV | BHV-1 |
|---|---|---|---|
| A6 | + | 0 | 0 |
| C1 | + | 0 | 0 |
| C2 | − | 55.8 + 4.6 | 75.5 + 3.5 |
| Vero | − | 50.8 + 6.2 | 65.0 + 5.6 |

* In this table, lines A6 and C1 are cellular lines transformed by the plasmid pCXN2/pHveCIg and expressing the chimeric protein PHveCIg whilst line C2 corresponds to a negative reference of this transformation not expressing the transgene and the Vero line to the initial cells sensitive to the viruses and used for production of the lines transformed by the different transgenes.

TABLE 9
Resistance of the transformed cellular lines to the PRV and
BHV-1 alphaherpesviruses

Number of lysis ranges observed

| Cellular line* | PRV | BHV-1 |
|---|---|---|
| 3-16 | 0 | 1 |
| V110 | 0 | 6 |
| Vero | 56 | 67 |

*In this table, line 3-16 is a cellular line transformed by the plasmid p CXN2/pVCC - pFc and line V110 is a cellular line transformed by the plasmid p CXN2/pV - pFc whilst the Vero line corresponds to the initial cells sensitive to the viruses and used for the production of cells transformed by the different transgenes.

1. Process for producing a mammal belonging to a non-human species rendered resistant by germinal transgenesis to an infection by an alphaherpesvirus, for which the polypeptide HveC or nectin-1 constitutes a functional receptor, characterized in that a transgene allowing the expression of a chimeric protein composed on the one hand of the extracellular domain of the nectin-1 or HveC or of one of its parts and on the other of the crystallisable fragment of an immunoglobulin, is introduced by insertion or homologous recombination in the genome of the cells constituting the germinal line of the mammal, in an appropriate system of expression.

2. Process according to claim 1, characterized in that the immunoglobulin is a gamma type immunoglobulin.

3. Process according to claim 1, characterized in that the nectin-1 or HveC and/or immunoglobulin belong to the homologous species.

4. Mammal belonging to a non-human species, characterized in that it has been rendered resistant by germinal transgenesis to an infection by an alphaherpesvirus for which the polypeptide HveC or nectin-1 constitutes a functional receptor by the effect of the expression of a chimeric protein composed on the one hand of the extracellular domain of the nectin-1 or HveC, preferably of the homologous species, and on the other hand of the crystallisable fragment of an immunoglobulin, notably a gamma type immunoglobulin, preferably of the homologous species.

5. Mammal according to claim 4, characterized in that the specific receptor of the alphaherpesvirus is a sub-part of the extracellular domain of nectin-1 or HveC.

6. Mammal according to claim 4, characterized in that it belongs to the porcine species and the alphaherpesvirus is the PRV virus.

7. Mammal according to claim 4, characterized in that it belongs to the bovine species and the alphaherpesvirus is the BHV-1 virus.

8. Mammal according to claim 4, characterized in that it contains in the genome of its cells a coding transgene for a chimeric protein composed on the one hand of the extracellular domain of nectin-1 or HveC or of one of its parts, and on the other of the crystallisable fragment of an immunoglobulin, in an appropriate expression system, this transgene having been inserted in the genome of the germinal line of one of its parents.

9. Genetic material such as semen or ooecyte or embryo essentially derived from the mammal according to claim 4.
